(19) United States
(12) Patent Application Publication
(10) Pub. No.: US 2021/0378580 A1
(43) Pub. Date: Dec. 9, 2021

(54) BRAIN WAVE MONITORING SYSTEM AND METHOD THEREOF

(71) Applicant: HippoScreen Neurotech Corp., Taipei City (TW)

(72) Inventors: Chang-Hsin WENG, Taipei City (TW); Wen-Hung LAN, Taipei City (TW); Chung-Lin HOU, Taipei City (TW)

(21) Appl. No.: 17/197,043

(22) Filed: Mar. 10, 2021

(30) Foreign Application Priority Data
Jun. 8, 2020 (TW) 109119125

(51) Int. Cl. A61B 5/369; A61B 5/00; A61B 5/384

(52) U.S. Cl. CPC A61B 5/369; A61B 5/742; A61B 5/7221; A61B 5/384; A61B 5/7257

(57) ABSTRACT

A brain wave monitoring system includes an electronic device and at least one electroencephalograph connected to the electronic device, each electroencephalograph includes 8 channels respectively connected to a corresponding electrode for receiving a brain wave signal. The electronic device scans a number of the at least one electroencephalograph and sequentially displays multiple channels of the at least one electroencephalograph according to an identifier of each of the at least one electroencephalograph. The brain wave monitoring system can automatically detect the number of electroencephalographs connected to the electronic device, and display the brain wave signals of all channels of all electroencephalographs according to the number of electroencephalographs and 8 channels of each electroencephalograph. The brain wave monitoring system can also prompt a guide message according to a channel impedance value and a signal quality index to assist a user improving a quality of the brain wave.

### FIELD OF THE INVENTION

The present invention relates to a brain wave monitoring technology, and more particularly to a brain wave monitoring system and method thereof.

### BACKGROUND OF THE INVENTION

Traditional electroencephalography is relatively large and heavy, and has a disadvantage of not being portable. Most electroencephalography apparatuses are used in hospitals, clinics and other professional medical places. Now the main medical trend is home care, for home patients who need the electroencephalography. In the limited home space, the disadvantages of the traditional electroencephalography being large size and heavy weight are more prominent. Also, the traditional electroencephalography is expensive. The traditional electroencephalography has 32 channels to measure brain wave signals of patients, and displays the 32 channels of brain wave signals at the same time. That is, when all the 32 channels are not needed, for example, if only 16 channels are used, the 32 channels will still be displayed and 16 of them will be idle, thereby causing waste of resources. In addition, the traditional electroencephalography can only measure the channel impedance, and cannot monitor the actual brain wave signal quality. The user lacks guidance information to assist in the operation, and can only obtain the usable brain wave signal quality through experience and wrong attempts. Therefore, how to improve the above-mentioned problems of the traditional electroencephalography is an important subject to be solved in the technical field.

### SUMMARY OF THE INVENTION

The present invention provides a brain wave monitoring system and a method thereof, which has advantages of small size and light weight, and can expand the number of brain wave meters according to requirements, so as to achieve convenience of carrying, expandability and reduce waste of resources.

The brain wave monitoring system provided by the present invention includes an electronic device and at least one electroencephalograph connected to the electronic device. Each of the at least one electroencephalograph includes 8 channels respectively connected to a corresponding electrode for receiving a brain wave signal. The electronic device scans a number of the at least one electroencephalograph and sequentially displays multiple channels of the at least one electroencephalograph according to an identifier of each of the at least one electroencephalograph.

In an embodiment of the present invention, the electronic device includes a processor, and the processor is used to measure channel impedance values of the multiple channels and output light signals of the multiple channels according to the channel impedance values, and to measure channel signal quality indexes of the multiple channels and output the light signals of the multiple channels according to the channel signal quality indexes.

In an embodiment of the present invention, when a channel impedance value of a channel is smaller than or equal to a preset impedance value, the electronic device displays a green light signal corresponding to the channel; and when the channel impedance value of the channel is larger than the preset impedance value, the electronic device displays a non-green light signal corresponding to the channel and prompts a guide message.

In an embodiment of the present invention, when a channel signal quality index of a channel is smaller than or equal to a preset signal quality index, the electronic device displays a green light signal corresponding to the channel; and when the channel signal quality index of the channel is larger than the preset signal quality index, the electronic device displays a non-green light signal corresponding to the channel and prompts a guide message.

In an embodiment of the present invention, the processor captures the brain wave signal of each channel in a time segment, converts the brain wave signal in the time segment into a power spectral density through fast Fourier transform, and sums up a power of a frequency band in the power spectral density to obtain the channel signal quality index, and determine whether the channel signal quality index of the channel is smaller than or equal to the preset signal quality index.

The brain wave monitoring method provided by the present invention is applied to a brain wave monitoring system including an electronic device and at least one electroencephalograph connected to the electronic device. The brain wave monitoring method includes steps of: scanning a number of the at least one electroencephalograph; and sequentially displaying multiple channels of the at least one electroencephalograph according to an identifier of each of the at least one electroencephalograph; wherein each of the at least one electroencephalograph includes 8 channels respectively connected to a corresponding electrode for receiving a brain wave signal.

In an embodiment of the present invention, the brain wave monitoring method further includes steps of: measuring channel impedance values of the multiple channels and outputting light signals of the multiple channels according to the channel impedance values; and measuring channel signal quality indexes of the multiple channels and outputting the light signals of the multiple channels according to the channel signal quality indexes.

In an embodiment of the present invention, the step of measuring channel signal quality indexes of the multiple channels and outputting the light signals of the multiple channels according to the channel signal quality indexes includes: capturing the brain wave signal of each channel in a time segment, converting the brain wave signal in the time segment into a power spectral density through fast Fourier transform, and summing up a power of a frequency band in the power spectral density to obtain a channel signal quality index, and determining whether the channel signal quality index is smaller than a preset signal quality index.

The brain wave monitoring system and method thereof provided by the present invention can not only achieve the convenience of carrying, expandability, and reduce waste of resources, but also measure the channel impedance and the channel signal quality to display the light signal of the channel status and guide how users can improve the quality of brain wave signals.

In order to make the above and other objects, features, and advantages of the present invention more comprehensible, embodiments are described below in detail with reference to the accompanying drawings, as follows.

### BRIEF DESCRIPTION OF THE DRAWINGS

FIG. 1 is a schematic diagram of a brain wave monitoring system provided by an embodiment of the present invention;

FIG. 2 is a first schematic diagram of a display interface provided by an embodiment of the present invention;

FIG. 3 is a second schematic diagram of a display interface provided by an embodiment of the present invention;

FIG. 4 is a flowchart of a brain wave monitoring method provided by an embodiment of the present invention;

FIG. 5 is a flowchart of measuring channel impedance values provided by an embodiment of the present invention; and

FIG. 6 is a flowchart of measuring channel signal quality indexes provided by an embodiment of the present invention.

### DETAILED DESCRIPTION OF PREFERRED EMBODIMENTS

Hereinafter, the present invention will be described in detail with drawings illustrating various embodiments of the present invention. However, the concept of the present invention may be embodied in many different forms and should not be construed as limitative of the exemplary embodiments set forth herein. In addition, the same reference number in the figures can be used to represent the similar elements.

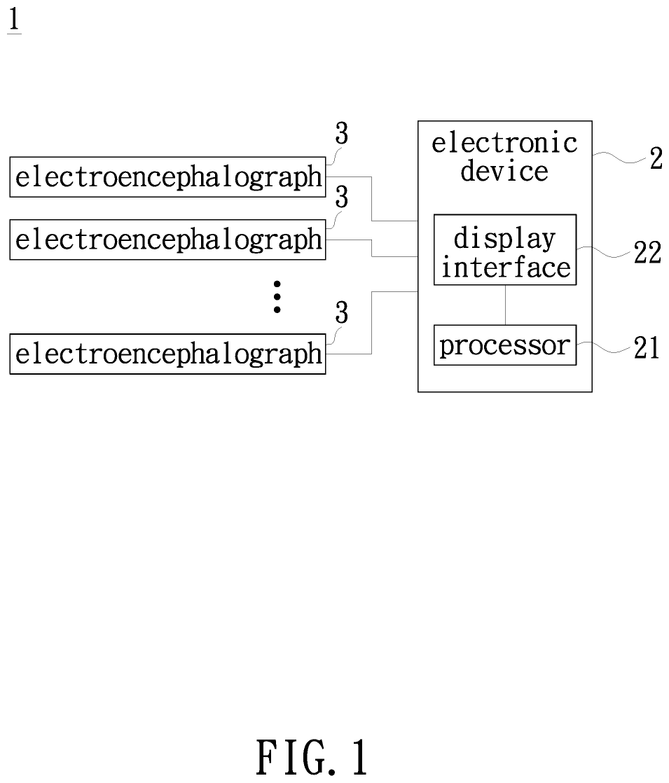

FIG. 1 is a schematic diagram of a brain wave monitoring system provided by an embodiment of the present invention. As shown in FIG. 1, the brain wave monitoring system 1 provided by the embodiment of the present invention includes an electronic device 2 and at least one electroencephalograph 3. The electronic device 2 may be a handheld device, such as a smart phone, a smart tablet, or a notebook that can be operated by an operator, and the notebook is only used as an example. In addition, the electronic device 2 includes a processor 21 and a display interface 22, wherein the processor 21 can be a microprocessor, a processing unit, or an arithmetic logic unit that processes data and instructions of the electronic device 2. The electroencephalograph 3 has 8 channels for connecting corresponding electrodes to receive patient's brain wave signals, and the electroencephalograph 3 is connected to the electronic device 2 through a universal serial bus (USB). Different connection interfaces which are used to connect the electroencephalograph with the electronic device can be selected by those skilled in the art according to actual needs, and the present invention is not limited herein. In addition, since the electroencephalograph of the embodiment of the present invention provides 8 channels to receive brain wave signals, those skilled in the art can increase the number of electroencephalographs according to medical needs to measure the complete brain wave signals of the patient.

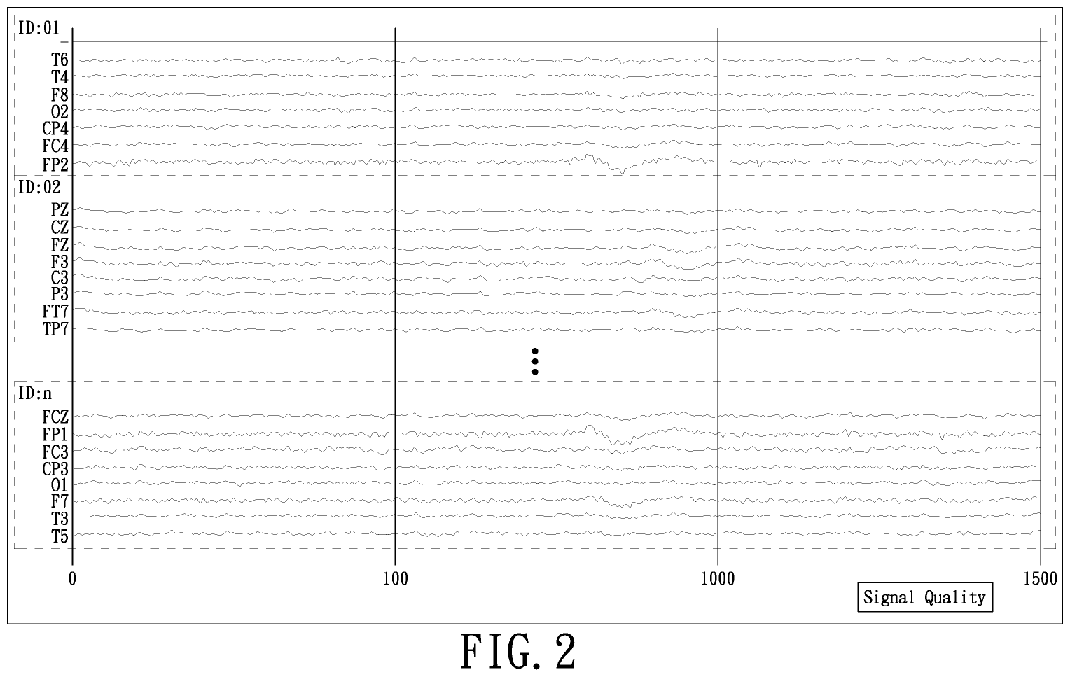

In the embodiment, when there are n electroencephalographs 3 connected to the electronic device 2, the electronic device 2 will scan n electroencephalographs 3, and the processor 21 of the electronic device 2 will perform a brain wave monitoring software to displays n*8 channels of brain wave signals in sequence on the display interface 22 according to identifiers (IDs) of n electroencephalographs 3, where n is a positive integer. As shown in FIG. 2, the numbers 01, 02, and n of the identifiers are only examples. The identifiers can also be a product serial number of the electroencephalograph or other codes that can be used as an identifier, and the present invention is not limited herein. In an example, when an electroencephalograph 3 is connected to the electronic device 2, the electronic device 2 scans the electroencephalograph 3, and the processor 21 of the electronic device 2 executes a brain wave monitoring software to display the brain wave signals of 8 (1*8) channels on the display interface 22 according to the identifier of the electroencephalograph 3. In another example, when three electroencephalographs 3 are connected to the electronic device 2, the electronic device 2 scans the three electroencephalographs 3, and the processor 21 of the electronic device 2 executes the brain wave monitoring software to sequentially displays the brain wave signals of 24 (3*8) channels on the display interface 22 according to the identifiers of the three electroencephalographs 3.

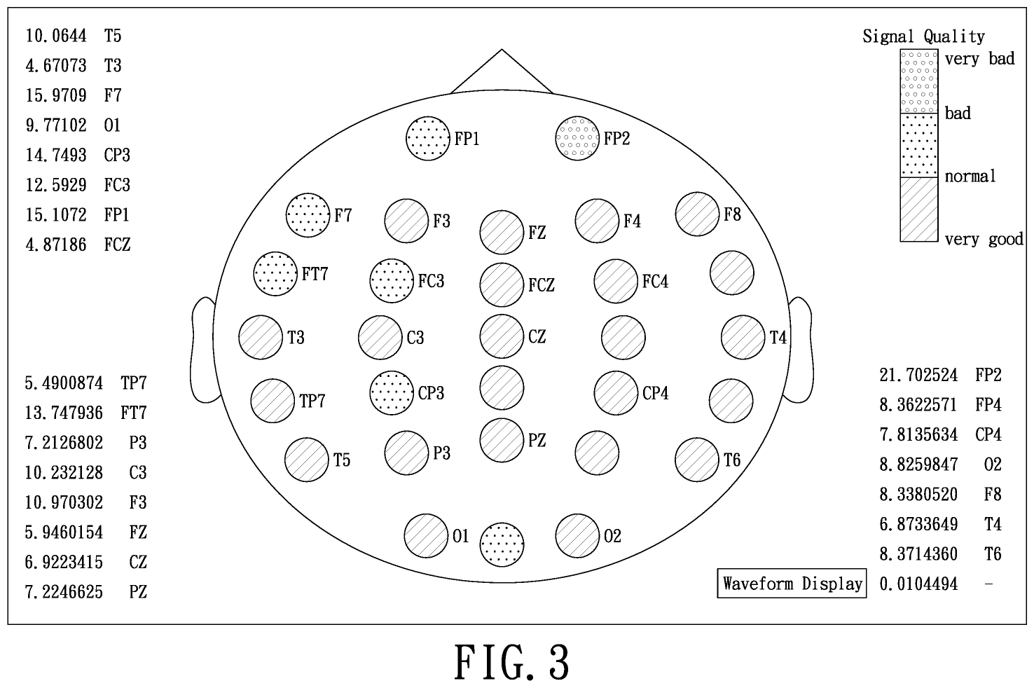

When the operator wants to view the quality of the brain wave signals of all channels, the operator can click the “Signal Quality” button at the bottom right of the display interface 22 to switch the screen on the display interface 22 to display all channels corresponding to the patient's brain position, as shown in FIG. 3. The light signals are divided into green lights and non-green lights. The green lights are represented by the “I” symbol to indicate that the channel impedance and the channel signal quality are good. The non-green lights are represented by the “⋅” or “•” symbols to indicate that the channel impedance and the channel signal quality are normal or poor, and can be orange light or red light. Those skilled in the art can customize the color of the light signals to represent different channel impedances and signal quality conditions. Therefore, the invention is not limited herein. In addition, when the operator wants to view the brain wave signals of all channels, the operator can click the “Waveform Display” button at the bottom right of the display interface 22 to switch the screen on the display interface 22 to display the brain wave signals of all channels corresponding to the patient's brain position.

The processor 21 of the electronic device 2 measures the channel impedance values of all channels and outputs the light signals of all channels according to the channel impedance values of all channels, so that the display interface 22 of the electronic device 2 displays the light signals of all channels. The measurement of the channel impedance values of all channels in the electroencephalograph is a technique well known to those skilled in the art, so it is not repeated herein. When a channel impedance value is smaller than or equal to a preset impedance value, the display interface 22 of the electronic device 2 will display a green light; and when the channel impedance value is larger than the preset impedance value, the display interface 22 of the electronic device 2 will display a non-green light and prompt a guide message. It could be noted that those skilled in the art can select the preset impedance value according to actual needs. However, the preset impedance value is preferably 10 kΩ. In an example, when a channel impedance value of a channel is smaller than or equal to the preset impedance value, the display interface 22 of the electronic device 2 will display the light signal of this channel as a green light, and when the channel impedance value of a channel is larger than the preset impedance value, the display interface 22 of the electronic device 2 will display the light signal of this channel as an orange light or a red light. And when the operator clicks the light signal of a channel on the display interface 22, the display interface 22 of the electronic device 2 will prompt a guide message, such as “please inject the gel again or check whether the electrode is adhered” to inform the operator to operate.

The processor 21 of the electronic device 2 measures the channel signal quality indexes of all channels and outputs the light signals of all channels according to the channel signal quality indexes of all channels, so that the display interface 22 of the electronic device 2 displays the light signals of all channels. The processor 21 of the electronic device 2 captures the brain wave signal of each channel in a time segment, converts the brain wave signal in the time segment into a power spectral density through fast Fourier transform, sums up a power of a frequency band in the power spectral density to obtain the channel signal quality index, and determines whether the channel signal quality index of the channel is smaller than or equal to the preset signal quality index. It could be noted that those skilled in the art can select a length of the time segment according to actual needs. However, the time period is preferably 3 seconds. When a channel signal quality index of a channel is smaller than or equal to the preset signal quality index, the display interface 22 of the electronic device 2 will display a green light, and when the channel signal quality index of the channel is larger than the preset signal quality index, the display interface 22 of the electronic device 2 will display a non-green light and prompt a guide message. In an example, when the channel signal quality index of a channel is smaller than or equal to the preset signal quality index, the display interface 22 of the electronic device 2 will display the light signal of this channel as a green light, and when the channel signal quality index of a channel is larger than the preset signal quality index, the display interface 22 of the electronic device 2 will display the light signal of this channel as an orange light or a red light. And when the operator clicks the light signal of this channel on the display interface 22, the display interface 22 of the electronic device 2 will prompt a guide message, such as “please relax the patient and check the position of the electrode wire” to inform the operator to operate.

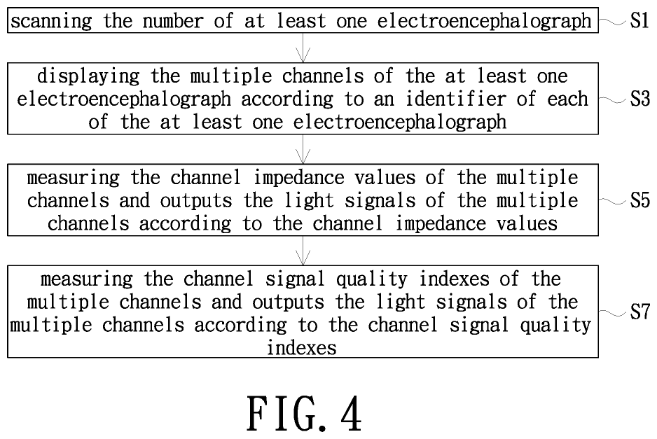

FIG. 4 is a flowchart of a brain wave monitoring method provided by an embodiment of the present invention. As shown in FIG. 4, the brain wave monitoring method provided by the present invention is applied to a brain wave monitoring system 1. The brain wave monitoring method is performed to include the following steps that, step S1: the processor 21 of the electronic device 2 scans the number of at least one electroencephalograph 3; step S3: the processor 21 of the electronic device 2 sequentially displays the multiple channels of the at least one electroencephalograph 3 according to an identifier of each of the at least one electroencephalograph 3; step S5: the processor 21 of the electronic device 2 measures the channel impedance values of the multiple channels and outputs the light signals of the multiple channels according to the channel impedance values; and step S7: the processor 21 of the electronic device 2 measures the channel signal quality indexes of the multiple channels and outputs the light signals of the multiple channels according to the channel signal quality indexes.

In step S1, when n electroencephalographs 3 are connected to the electronic device 2, the electronic device 2 scans the identifiers (IDs) of n electroencephalographs 3 to identify the number of n electroencephalograms 3, where n is a positive integer.

In step S3, the processor 21 of the electronic device 2 executes a brain wave monitoring software to sequentially display n*8 channels of brain wave signals on the display interface 22 according to the identifiers (IDs) of n electroencephalographs 3, as shown in FIG. 2.

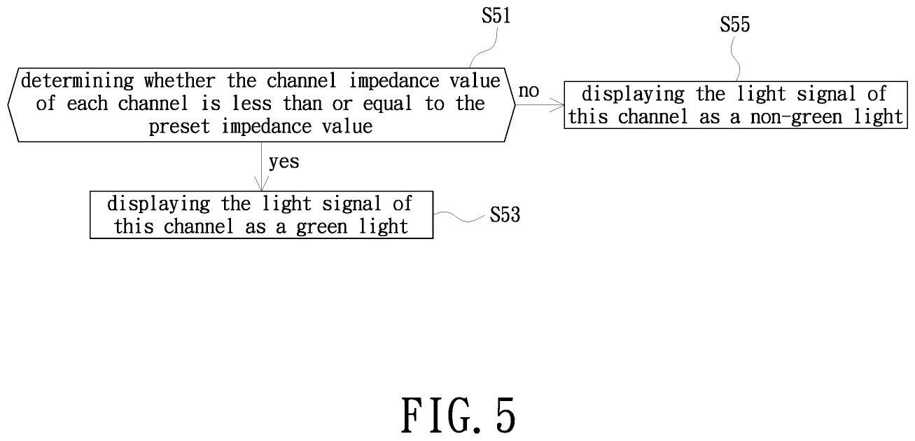

In step S5, the processor 21 of the electronic device 2 measures the channel impedance value of each channel of n electroencephalographs 3, as shown in FIG. 5, and executes steps which includes, step S51: determining whether the channel impedance value of each channel is less than or equal to the preset impedance value; step S53: when the channel impedance value of a channel is less than or equal to the preset impedance value, the display interface 22 of the electronic device 2 displays the light signal of this channel as a green light; and S55: When the channel impedance value of a channel is greater than the preset impedance value, the display interface 22 of the electronic device 2 displays the light signal of this channel as a non-green light. Then, the processor 21 of the electronic device 2 outputs the light signals of all the channels according to the channel impedance values of all the channels, so that the display interface 22 of the electronic device 2 displays the light signals of all the channels.

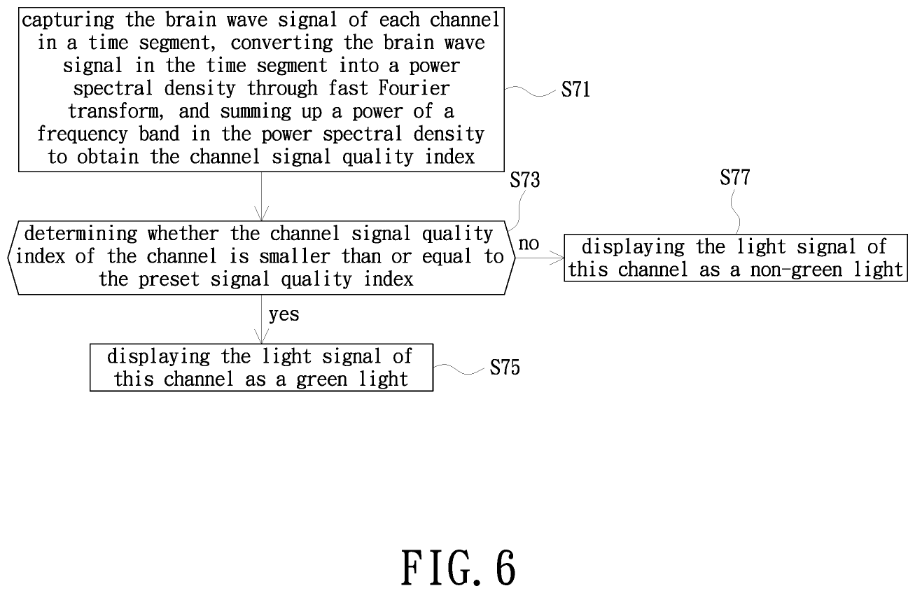

In step S7, as shown in FIG. 6, the processor 21 of the electronic device 2 performs the steps that includes: step S71: capturing the brain wave signal of each channel in a time segment, converting the brain wave signal in the time segment into a power spectral density through fast Fourier transform, and summing up a power of a frequency band in the power spectral density to obtain the channel signal quality index; S73: determining whether the channel signal quality index of the channel is smaller than or equal to the preset signal quality index; step S75: when the channel signal quality index of a channel is smaller than or equal to the preset signal quality index, the display interface 22 of the electronic device 2 displays the light signal of this channel as a green light; and step S77: when the channel signal quality index of a channel is larger than the preset signal quality index, the display interface 22 of the electronic device 2 displays the light signal of this channel as a non-green light. Then, the processor 21 of the electronic device 2 outputs the light signals of all the channels according to the channel signal quality indexes of all the channels, so that the display interface 22 of the electronic device 2 displays the light signals of all the channels.

In summary, the brain wave monitoring system and method thereof provided by the present invention has advantages of small size and light weight, and expands the number of electroencephalographs according to requirements, so as to achieve convenience of carrying, expandability and reduce waste of resources, and also measures the channel impedance and the channel signal quality to display the light signal of the channel status and guide how users can improve the quality of brain wave signals.

Although the present invention has been disclosed as above with the embodiments, it is not intended to limit the present invention. Those ordinarily skilled in the art may make some modifications and retouching without departing from the spirit and scope of the present invention. Therefore, the protection scope of the present invention shall be determined by the scope of the attached claims.

What is claimed is:

1. A brain wave monitoring system, comprising:
an electronic device; and
at least one electroencephalograph connected to the electronic device, each of the at least one electroencephalograph including 8 channels respectively connected to a corresponding electrode for receiving a brain wave signal;
wherein the electronic device scans a number of the at least one electroencephalograph and sequentially displays multiple channels of the at least one electroencephalograph according to an identifier of each of the at least one electroencephalograph.

2. The brain wave monitoring system as claimed in claim 1, wherein the electronic device includes a processor; and the processor is used to measure channel impedance values of the multiple channels and output light signals of the multiple channels according to the channel impedance values, and to measure channel signal quality indexes of the multiple channels and output the light signals of the multiple channels according to the channel signal quality indexes.

3. The brain wave monitoring system as claimed in claim 2, wherein when a channel impedance value of a channel is smaller than or equal to a preset impedance value, the electronic device displays a green light signal corresponding to the channel; and when the channel impedance value of the channel is larger than the preset impedance value, the electronic device displays a non-green light signal corresponding to the channel and prompts a guide message.

4. The brain wave monitoring system as claimed in claim 2, wherein when a channel signal quality index of a channel is smaller than or equal to a preset signal quality index, the electronic device displays a green light signal corresponding to the channel; and when the channel signal quality index of the channel is larger than the preset signal quality index, the electronic device displays a non-green light signal corresponding to the channel and prompts a guide message.

5. The brain wave monitoring system as claimed in claim 4, wherein the processor captures the brain wave signal of each channel in a time segment, converts the brain wave signal in the time segment into a power spectral density through fast Fourier transform, and sums up a power of a frequency band in the power spectral density to obtain the channel signal quality index, and determine whether the channel signal quality index of the channel is smaller than or equal to the preset signal quality index.

6. A brain wave monitoring method applied to a brain wave monitoring system including an electronic device and at least one electroencephalograph connected to the electronic device, the brain wave monitoring method comprising:
scanning a number of the at least one electroencephalograph; and
sequentially displaying multiple channels of the at least one electroencephalograph according to an identifier of each of the at least one electroencephalograph;
wherein each of the at least one electroencephalograph includes 8 channels respectively connected to a corresponding electrode for receiving a brain wave signal.

7. The brain wave monitoring method as claimed in claim 6, further comprising:
measuring channel impedance values of the multiple channels and outputting light signals of the multiple channels according to the channel impedance values; and
measuring channel signal quality indexes of the multiple channels and outputting the light signals of the multiple channels according to the channel signal quality indexes.

8. The brain wave monitoring method as claimed in claim 7, wherein when a channel impedance value of a channel is smaller than or equal to a preset impedance value, the electronic device displays a green light signal corresponding to the channel; and when the channel impedance value of the channel is larger than the preset impedance value, the electronic device displays a non-green light signal corresponding to the channel and prompts a guide message.

9. The brain wave monitoring method as claimed in claim 7, wherein the step of measuring channel signal quality indexes of the multiple channels and outputting the light signals of the multiple channels according to the channel signal quality indexes includes:
capturing the brain wave signal of each channel in a time segment, converting the brain wave signal in the time segment into a power spectral density through fast Fourier transform, and summing up a power of a frequency band in the power spectral density to obtain a channel signal quality index, and determining whether the channel signal quality index is smaller than a preset signal quality index.

10. The brain wave monitoring method as claimed in claim 9, wherein when the channel signal quality index of a channel is smaller than or equal to the preset signal quality index, the electronic device displays a green light signal corresponding to the channel; and when the channel signal quality index of the channel is larger than the preset signal quality index, the electronic device displays a non-green light signal corresponding to the channel and prompts a guide message.
